(19) United States
(12) Patent Application Publication
(10) Pub. No.: US 2014/0194951 A1
(43) Pub. Date: Jul. 10, 2014

(54) ELECTRICAL STIMULATION APPARATUS AND METHOD

(71) Applicant: INDUSTRIAL TECHNOLOGY RESEARCH INSTITUTE, Hsinchu (TW)

(72) Inventors: Cihun-Siyong Gong, Kaohsiung City (TW); Muh-Tian Shiue, Hsinchu City (TW)

(73) Assignee: Industrial Technology Research Institute, Hsinchu (TW)

(21) Appl. No.: 13/851,100

(22) Filed: Mar. 27, 2013

(30) Foreign Application Priority Data
Jan. 7, 2013 (TW) 102100461

(51) Int. Cl. A61N 1/372

(52) U.S. Cl. CPC A61N 1/37252
USPC 607/59; 607/62

(57) ABSTRACT

An electrical stimulation apparatus and an electrical stimulation method are provided. The electrical stimulation apparatus may include an electrode unit, a measurement unit and a stimulation unit. The electrode unit is used for contacting a tissue of interest (target tissue). The measurement unit is coupled to the electrode unit. The measurement unit measure a tissue characteristic of the target tissue. The stimulation unit is coupled to the measurement unit and the electrode unit. The stimulation unit stimulates the target tissue through the electrode unit by using an electrical stimulation signal, and the stimulation unit determines an amount of charge of the electrical stimulation signal according to the tissue characteristic measured by the measurement unit.

### CROSS-REFERENCE TO RELATED APPLICATION

This application claims the priority benefit of Taiwan application serial no. 102100461, filed on Jan. 7, 2013. The entirety of the above-mentioned patent application is hereby incorporated by reference herein and made a part of this specification.

### BACKGROUND

1. Technical Field

The technical field relates to an electronic apparatus, an electrical stimulation apparatus and an electrical stimulation method.

2. Related Art

In the field of biomedical sciences, implantable electronic devices such as the implantable neural stimulator (INS) have been widely adopted to monitor internal organs, tissues, neural or cell conditions, and/or restore lost physiological functions. Among the various types of implantable electronic devices, the electrical stimulation apparatus can activate a biological tissue (referred to here as a target tissue) by injecting and accumulating charge in the target tissue. However, the amount of charge (i.e. stimulus resolution) being injected into the target tissue must be accurate.

Generally speaking, the electrical stimulation apparatus can be broadly classified into the voltage-controlled scenario (VCS), the current-controlled scenario (CCS), and the switched capacitor array scenario (SCS). The energy efficiency of the conventional CCS electrical stimulation apparatus is poor, and the energy efficiency of the VCS electrical stimulation apparatus is preferable. A high energy efficiency increases the lifespan of the implantable electronic device.

### SUMMARY

The disclosure provides an electrical stimulation apparatus, may include an electrode unit, a measurement unit, and a stimulation unit. The electrode unit may be used for contacting a biological tissue (referred to hereafter as a target tissue). The measurement unit may be coupled to the electrode unit. The measurement unit measures a tissue characteristic of the target tissue through the electrode unit. The stimulation unit may be coupled to the electrode unit and the measurement unit. The stimulation unit stimulates the target tissue through the electrode unit by using an electrical stimulation signal, and determines an amount of charge of the electrical stimulation signal according to the tissue characteristic measured by the measurement unit.

The disclosure provides an electrical stimulation method, may include: disposing an electrode unit contacting a target tissue of a biological body; by using a measurement unit, measuring a tissue characteristic of the target tissue through the electrode unit; and by using a stimulation unit, stimulating the target tissue through the electrode unit by using an electrical stimulation signal, wherein an amount of charge of the electrical stimulation signal is determined by the stimulation unit according to the tissue characteristic measured by the measurement unit.

In summary, the disclosure provides an electrical stimulation apparatus and an electrical stimulation method. The measuring unit measures the tissue characteristic of the target tissue, and the tissue characteristic is fed back to the stimulation unit. The stimulation unit can determine the amount of charge of the electrical stimulation signal according to the tissue characteristic measured by the measurement unit, and stimulate the target tissue by using the electrical stimulation signal. Therefore, the electrical stimulation apparatus and electrical stimulation method provided by the disclosure can satisfy the stimulus resolution (charge accuracy) requirements. In some embodiments, the electrical stimulation apparatus provided by the disclosure can use a voltage source to implement the voltage-controlled scenario, to meet both the high energy efficiency and low circuit area requirements.

Several exemplary embodiments accompanied with figures are described in below to further describe the disclosure in details.

### BRIEF DESCRIPTION OF THE DRAWINGS

The accompanying drawings constituting a part of this specification are incorporated herein to provide a further understanding of the disclosure. Here, the drawings illustrate embodiments of the disclosure and, together with the description, serve to explain the principles of the embodiments.

FIG. 1 is a circuit block diagram of an electrical stimulation apparatus according to an embodiment of the disclosure.

FIG. 2 is a voltage curve diagram of an electrical stimulation signal according to an embodiment of the disclosure.

FIG. 3 is a current waveform diagram outputted by a stimulation unit according to an embodiment of the disclosure, when the electrical stimulation signal of FIG. 2 is applied and a time constant of a target tissue is longer than a period of the electrical stimulation signal.

FIG. 4 is a current waveform diagram outputted by a stimulation unit according to an embodiment of the disclosure, when the electrical stimulation signal of FIG. 2 is applied and a time constant of the target tissue is approximately equal to a period of the electrical stimulation signal.

FIG. 5 is a current waveform diagram outputted by a stimulation unit according to an embodiment of the disclosure, when the electrical stimulation signal of FIG. 2 is applied and a time constant of a target tissue is shorter than a period of the electrical stimulation signal.

FIG. 6 is a flow diagram of an electrical stimulation method according to an embodiment of the disclosure.

FIG. 7 is a circuit block diagram of the measurement unit and the electrode unit depicted in FIG. 1 according to an embodiment of the disclosure.

FIG. 8 is a flow diagram of Step S620 in FIG. 6 according to an embodiment of the disclosure.

FIG. 9 is a circuit block diagram of the measurement unit and the electrode unit depicted in FIG. 1 according to another embodiment of the disclosure.

FIG. 10 is a circuit block diagram of the electrical stimulation apparatus depicted in FIG. 1 according to an embodiment of the disclosure.

FIG. 11 is a flow diagram of Step S630 in FIG. 6 according to an embodiment of the disclosure.

FIG. 12 is a circuit block diagram of the stimulation driver circuit depicted in FIG. 10 according to an embodiment of the disclosure.

### DESCRIPTION OF EMBODIMENTS

The terms “connected,” “coupled,” and “mounted” and variations thereof herein (including the claims) are used broadly and encompass direct and indirect connections, couplings, and mountings. For example, if the disclosure describes a first apparatus being coupled to a second apparatus, the first apparatus can be directly connected to the second apparatus, or the first apparatus can be indirectly connected to the second apparatus through other devices or by a certain coupling means.

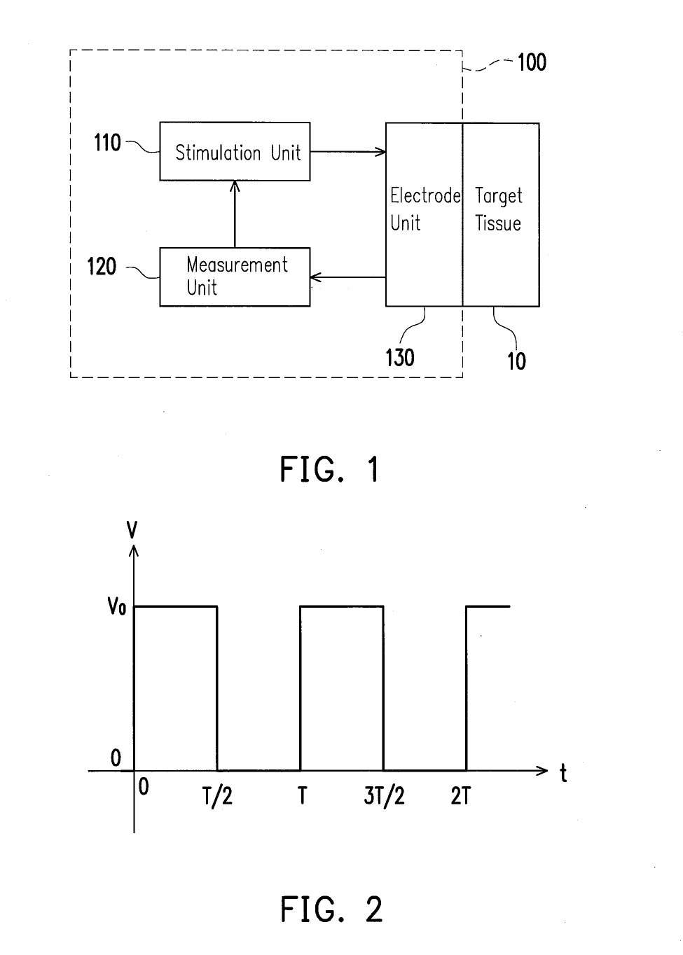

FIG. 1 is a circuit block diagram of an electrical stimulation apparatus according to an embodiment of the disclosure. With reference to FIG. 1, an electrical stimulation apparatus 100 includes a stimulation unit 110, a measurement unit 120, and an electrode unit 130. The stimulation unit 110 is coupled to the measurement unit 120 and the electrode unit 130. The measurement unit 120 is coupled to the electrode unit 130. The electrode unit 130 is used for contacting a biological tissue (referred to hereafter as a target tissue 10). According to different applications, the target tissue 10 may include a neural tissue, a cell tissue, or other biological (organic) tissues. The electrode unit 130 contacts the target tissue 10 through one or a plurality of electrodes (details described later in the disclosure).

The stimulation unit 110 is not limited to being implemented in the voltage-controlled scenario (VCS). In other embodiments, the stimulation unit 110 may also be implemented in the current-controlled scenario (CCS) or the switched capacitor array scenario (SCS). The stimulation unit 110 can stimulate the target tissue 10 through the electrode unit 130 by using an electrical stimulation signal having a pulse width, so as to inject charge into the target tissue 10. For example, the stimulation unit 110 can output an electrical stimulation signal having a pulse width of ½ period T to the target tissue 10. FIG. 2 is a voltage curve diagram of an electrical stimulation signal according to an embodiment of the disclosure. In FIG. 2, the horizontal axis represents the time t, and the vertical axis represents the voltage V (units of volt). Assume here that the stimulation unit 110 can output an electrical stimulation signal Vo having a plurality of pulses to the target tissue 10, so as to inject charge into the target tissue 10. However, different target tissues have different tissue characteristics (e.g., impedance, time constant, or other characteristics).

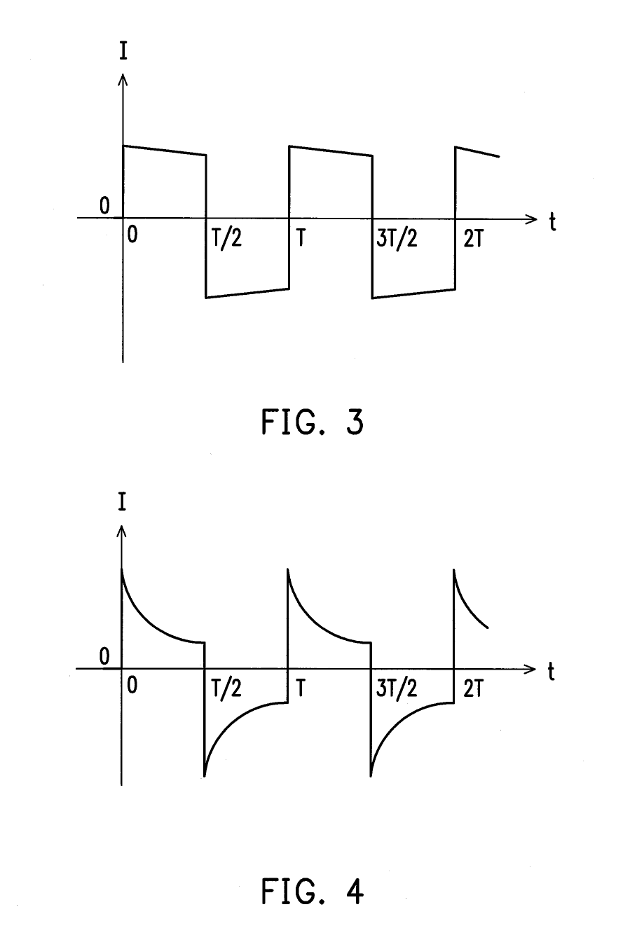

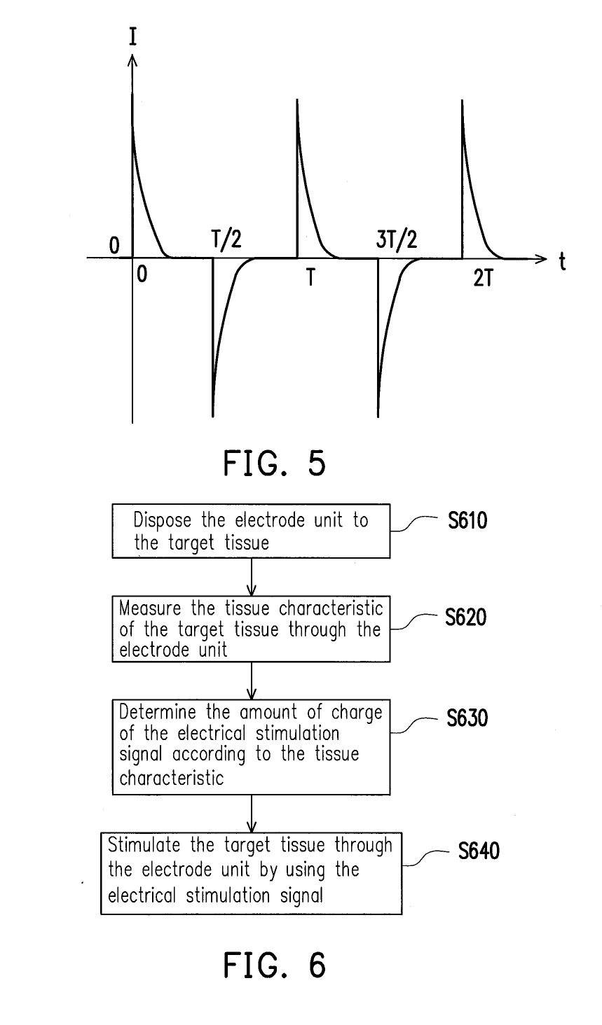

For example, FIG. 3 is a current waveform diagram outputted by the stimulation unit 110 according to an embodiment of the disclosure, when the electrical stimulation signal of FIG. 2 is applied and the time constant of the target tissue 10 is longer than the period T of the electrical stimulation signal. In FIG. 3, the horizontal axis represents the time t, and the vertical axis represents the current I (units of ampere). FIG. 4 is a current waveform diagram outputted by the stimulation unit 110 according to an embodiment of the disclosure, when the electrical stimulation signal of FIG. 2 is applied and the time constant of the target tissue 10 is approximately equal to the period T of the electrical stimulation signal. In FIG. 4, the horizontal axis represents the time t, and the vertical axis represents the current I. FIG. 5 is a current waveform diagram outputted by the stimulation unit 110 according to an embodiment of the disclosure, when the electrical stimulation signal of FIG. 2 is applied and the time constant of the target tissue 10 is shorter than the period T of the electrical stimulation signal. In FIG. 5, the horizontal axis represents the time t, and the vertical axis represents the current I. As shown in FIGS. 3-5, different target tissues have different tissue characteristics.

When the pulse width is fixed, the conventional VCS electrical stimulation apparatus controls an amount of output charge by adjusting a level of the electrical stimulation signal Vo. To accurately control the amount of output charge to suit the different tissue characteristics of different target tissues, the conventional VCS electrical stimulation apparatus requires a user to adjust the level of the electrical stimulation signal Vo.

FIG. 6 is a flow diagram of an electrical stimulation method according to an embodiment of the disclosure. With reference to FIGS. 1 and 6, in Step S610, the electrode unit 130 is disposed to contact the target tissue 10. In Step S620, the measurement unit 120 measures a tissue characteristic of the target tissue 10 through the electrode unit 130, and then feeds back the measured tissue characteristic to the stimulation unit 110. In Step S630, the stimulation unit 110 determines an amount of charge of the electrical stimulation signal according to the tissue characteristic measured by the measurement unit 120. For example, Step S630 can determine the amount of charge by determining a pulse width of the electrical stimulation signal. In Step S640, the stimulation unit 110 stimulates the target tissue 10 through the electrode unit 130 by using the electrical stimulation signal (with the pulse width determined/adjusted in Step S630), so as to inject charge into the target tissue 10. Since the stimulation unit 110 determines the pulse width of the electrical stimulation signal according to the tissue characteristic measured by the measurement unit 120, the stimulation unit 110 can accumulate an accurate amount of charge in the target tissue 10 automatically according to the differences of the target tissues. The accurate charge amount can activate the target tissue 10.

For example, in some embodiments, the stimulation unit 110 has a plurality of stimulation models. In Step S630, the stimulation models provided by the stimulation unit 110 has different current-time characteristics, such as the stimulation models shown in FIGS. 3-5, or other stimulation models derived from FIGS. 3-5. According to the tissue characteristic measured by the measurement unit 120, in Step S630, the stimulation unit 110 can select a target model from the stimulation models. For example, assuming the tissue characteristic includes an impedance or a time constant of the target tissue 10, the stimulation unit 110 can select a target model having an impedance or time constant closest to the target tissue 10 according to the impedance or time constant of the target tissue 10. According to the target model, the stimulation unit 110 can convert a target charge value to a pulse width (since charge Q=I*t, in which I is the output current, and t is time/pulse width). Therefore, the stimulation unit 110 can control the pulse width of the electrical stimulation signal according to the pulse width (e.g., controlling the pulse width of the electrical stimulation signal Vo shown in FIG. 2).

For example, in another embodiment, the stimulation unit 110 has a plurality of look-up tables. In Step S630, the look-up tables provided by the stimulation unit 110 respectively correspond to different tissue characteristics. According to the tissue characteristic measured by the measurement unit 120, the stimulation unit 110 can select a target look-up table from the plurality of look-up tables. According to the target look-up table, the stimulation unit 110 can convert the target charge value into the pulse width. Moreover, the stimulation unit 110 can control the pulse width of the electrical stimulation signal according to the pulse width. The implementation details of this embodiment are detailed later in the disclosure.

Therefore, the electrical stimulation apparatus 100 of the present embodiment can satisfy various stimulus resolution (charge accuracy) requirements, and output accurate amounts of charge to the target tissue 10. In the electrical stimulation apparatus 100 of the present embodiment, less voltage levels are needed for the electrical stimulation signal Vo (a single voltage level is possible). The electrical stimulation apparatus 100 of the present embodiment can drastically reduce the circuit area and can be implanted into a biological body.

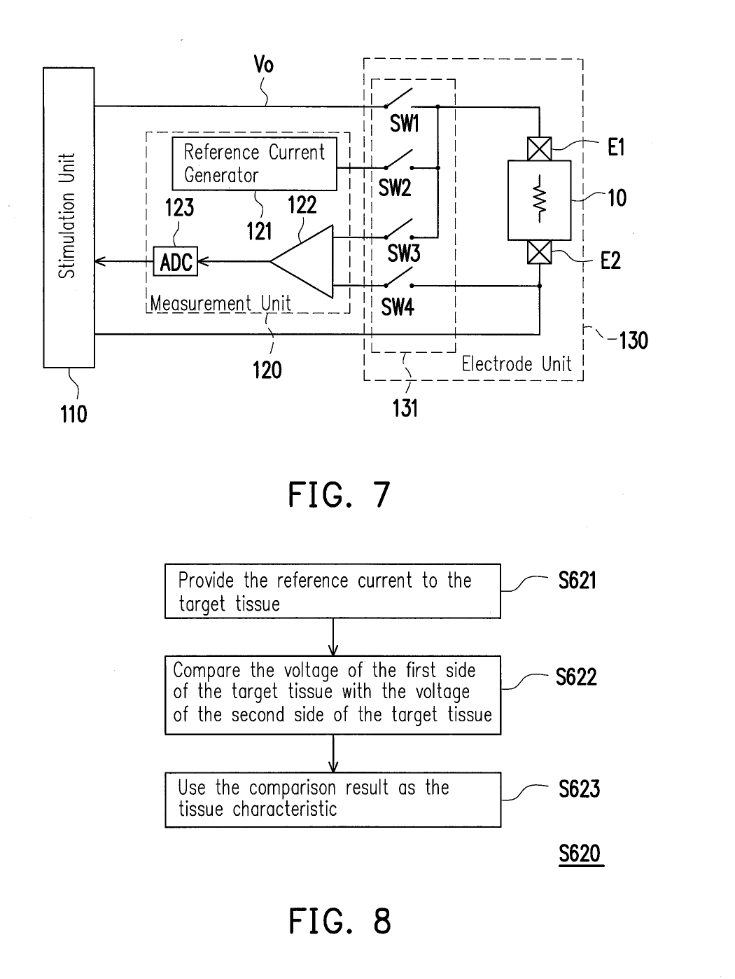

FIG. 7 is a circuit block diagram of the measurement unit 120 and the electrode unit 130 depicted in FIG. 1 according to an embodiment of the disclosure. The embodiment illustrated by FIG. 7 can be derived by referring to the related description of FIGS. 1-6. With reference to FIG. 7, the electrode unit 130 includes a first electrode E1, a second electrode E2, and a switching unit 131. The first electrode E1 is coupled to a first side of the target tissue 10, and the second electrode E2 is coupled to a second side of the target tissue 10. The first side and the second side may be opposite sides of the target tissue 10. The switching unit 131 is coupled to the first electrode E1, the second electrode E2, the measurement unit 120, and the stimulation unit 110. In a measurement period, the switching unit 131 couples the first electrode E1 and the second electrode E2 to the measurement unit 120. The measurement unit 120 can measure the tissue characteristic of the target tissue 10 in the measurement period through the electrode unit 130. In a stimulation period after the measurement period has ended, the switching unit 131 couples the first electrode E1 and the second electrode E2 to the stimulation unit 110. In the stimulation period, the stimulation unit 110 can determine the pulse width of the electrical stimulation signal Vo according to the tissue characteristic measured by the measurement unit 120, and stimulate the target tissue 10 through the electrode unit 130 by using the electrical stimulation signal Vo.

The measurement unit 120 and the switching unit 131 can be implemented by any methods. For example, in the present embodiment, the switching unit 131 includes a first switch SW1, a second switch SW2, a third switch SW3, and a fourth switch SW4. The measurement unit 120 includes a reference current generator 121, a voltage comparator 122, and an analog-to-digital converter (ADC) 123. The voltage comparator 122 may include an instrumentation amplifier, an operation amplifier, or an error amplifier.

The first switch SW1 has a first terminal coupled to an output terminal of the electrical stimulation signal from the stimulation unit 110, and a second terminal coupled to the first electrode E1. The second switch SW2 has a first terminal coupled to an output terminal of the reference current generator 121 of the measurement unit 120, and a second terminal coupled to the first electrode E1. The third switch SW3 has a first terminal coupled to a first input terminal of the voltage comparator 122 of the measurement unit 120, and a second terminal coupled to the first electrode E1. A fourth switch SW1 has a first terminal coupled to a second input terminal of the voltage comparator 122, and a second terminal coupled to the second electrode E2. In the present embodiment, the second electrode E2 is also coupled to the stimulation unit 110. In other embodiments, the second electrode E2 can be coupled to a ground voltage.

FIG. 8 is a flow diagram of Step S620 in FIG. 6 according to an embodiment of the disclosure. Please refer to FIGS. 7 and 8. In the measurement period, the second switch SW2, the third switch SW3, and the fourth switch SW4 are turned on, and the first switch SW1 is turned off. The reference current generator 121 provides a reference current to the target tissue 10 through the second switch SW2 and the first electrode E1 of the electrode unit 130 (Step S621). Since the target tissue 10 has an impedance, the reference current flowing to the target tissue 10 causes two sides of the target tissue 10 to have a potential difference. The first input terminal (e.g. a non-inverting input terminal) of the voltage comparator 122 is coupled to the first side of the target tissue 10 through the third switch SW3 and the first electrode E1 of the electrode unit 130. The second input terminal (e.g. an inverting input terminal) of the voltage comparator 122 is coupled to the second side of the target tissue 10 through the fourth switch SW4 and the second electrode E2 of the electrode unit 130. The voltage comparator 122 may be connected in a closed loop wiring state.

An input terminal of the ADC 123 is coupled to an output terminal of the voltage comparator 122. An output terminal of the ADC 123 is coupled to the stimulation unit 110. The ADC 123 can convert an analog output of the voltage comparator 122 into a digital code. The stimulation unit 110 can measure/compare the potential difference of two sides of the target tissue 10 in the measurement period by using the voltage comparator 122 (Step S622). Therefore, in some embodiments, the stimulation unit 110 in Step S623 can use the potential difference obtained in Step S622 to serve as the tissue characteristic of the target tissue 10. In the present embodiment, since resistance R=V/I (where V is the potential difference of two sides of the target tissue 10, and I is the reference current flowing to the target tissue 10 provided by the reference current generator 121), the stimulation unit 110 can obtain the impedance of the target tissue 10 in Step S623, to serve as the tissue characteristic of the target tissue 10.

In the stimulation period after the measurement period has ended, the first switch SW1 is turned on, and the second switch SW2, the third switch SW3, and the fourth switch SW4 are turned off. The stimulation unit 110 can transmit the electrical stimulation signal Vo with the modulated pulse width to the target tissue 10 through the first switch SW1 and the first electrode E1. Since the stimulation unit 110 has obtained the impedance of the target tissue 10, in the stimulation period, the stimulation unit 110 can determine the pulse width of the electrical stimulation signal Vo according to the impedance of the target tissue 10 (further elaboration later in the disclosure).

However, the implementation methods of the measurement unit 120 and the switching unit 131 are not limited by FIG. 7. For example, in another embodiment, the third switch SW3 and the fourth switch SW4 may be replaced by a first conductive line and a second conductive line. The first conductive line has a first end and a second end respectively coupled to the first input terminal of the voltage comparator 122 of the measurement unit 120 and the first electrode E1. The second conductive line has a first end and a second end respectively coupled to the second input terminal of the voltage comparator 122 and the second electrode E2.

In other embodiments for example, when the feedback terminal of the stimulation unit 110 is the analog input terminal, the ADC 123 can be omitted. When the ADC 123 is omitted, the output terminal of the voltage comparator 122 is coupled to the stimulation unit 110.

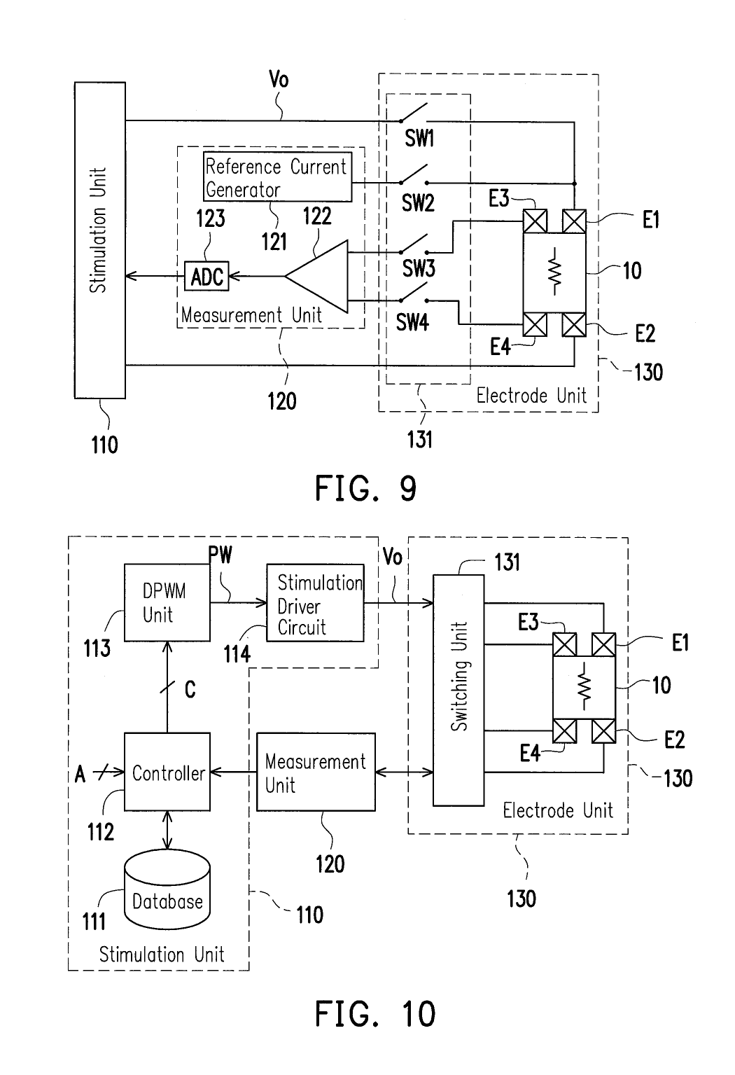

In another example, FIG. 9 is a circuit block diagram of the measurement unit 120 and the electrode unit 130 depicted in FIG. 1 according to another embodiment of the disclosure. The embodiment illustrated by FIG. 9 can be derived by referring to the related description of FIGS. 1-8. With reference to FIG. 9, the electrode unit 130 includes a first electrode E1, a second electrode E2, a third electrode E3, a fourth electrode E4, and a switching unit 131. The first electrode E1 is coupled to a first side of the target tissue 10, and the second electrode E2 is coupled to a second side of the target tissue 10. The first side and the second side may be opposite sides of the target tissue 10. The third electrode E3 is coupled to a third side of the target tissue 10, and the fourth electrode E4 is coupled to fourth side of the target tissue 10. The third side and the fourth side may be opposite sides of the target tissue 10. The switching unit 131 is coupled to the first electrode E1, the second electrode E2, the third electrode E3, the fourth electrode E4, the measurement unit 120, and the stimulation unit 110.

In the measurement period, the switching unit 131 couples the first electrode E1, the third electrode E3, and the fourth electrode E4 to the measurement unit 120. The measurement unit 120 can measure the tissue characteristic of the target tissue 10 in the measurement period through the electrode unit 130. In the stimulation period after the measurement period has ended, the switching unit 131 couples the first electrode E1 to the stimulation unit 110, and couples the second electrode E2 to the ground voltage or the stimulation unit 110. In the stimulation period, the stimulation unit 110 can determine the pulse width of the electrical stimulation signal Vo according to the tissue characteristic measured by the measurement unit 120, and stimulate the target tissue 10 through the electrode unit 130 by using the electrical stimulation signal Vo.

The measurement 120 and the switching unit 131 may be implemented by any methods, such as by reference to the related description of FIGS. 7 and 8, and thus further elaboration thereof is omitted. A difference compared to the embodiment depicted in FIG. 7 is that, in the embodiment of FIG. 9, the second terminal of the third switch SW3 is coupled to the third electrode E3, and the second terminal of the fourth switch SW4 is coupled to the fourth electrode E4.

FIG. 10 is a circuit block diagram of the electrical stimulation apparatus depicted in FIG. 1 according to an embodiment of the disclosure. The embodiment illustrated by FIG. 10 can be derived by referring to the related description of FIGS. 1-9. With reference to FIG. 10, the stimulation unit 110 includes a database 111, a controller 112, a digital pulse width modulation (DPWM) unit 113, and a stimulation driver circuit 114.

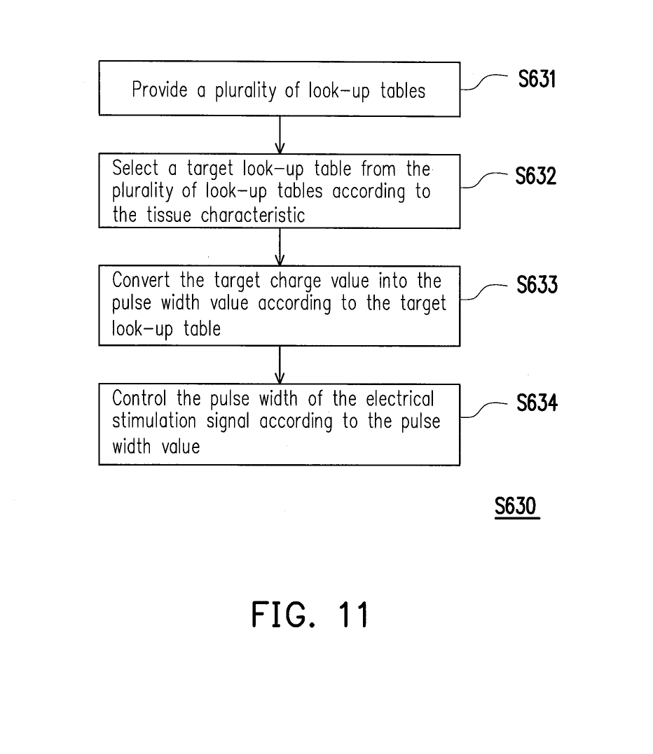

FIG. 11 is a flow diagram of Step S630 in FIG. 6 according to an embodiment of the disclosure. Please refer to FIGS. 10 and 11. The database 111 can provide a plurality of look-up tables (Step S631). Different look-up tables respectively correspond to different tissue characteristics. For example, Table 1 is one of the look-up tables corresponding to 10 KΩ (the impedance of the target tissue 10 plus the impedances of the electrodes E3 and E4), and Table 2 is another one of the look-up tables corresponding to 100 KΩ (the impedance of the target tissue 10 plus the impedances of the electrodes E3 and E4). The other look-up tables can be derived by referring to the related description of Tables 1 and 2. The contents of the different look-up tables in the database 111 may be set according to the design of a product.

TABLE 1
Exemplary Look-up Table Corresponding to 10 KΩ

| Target Charge Value | | | | | | | | | | | | | | |
|---|---|---|---|---|---|---|---|---|---|---|---|---|---|---|
| A[3:0] | | | | | Pulse Width C[9:0] | | | | | | | | | |
| code | A₃ | A₂ | A₁ | A₀ | C₉ | C₈ | C₇ | C₆ | C₅ | C₄ | C₃ | C₂ | C₁ | C₀ |
| 0 | 0 | 0 | 0 | 0 | 0 | 0 | 0 | 0 | 1 | 0 | 1 | 0 | 1 | 1 |
| 1 | 0 | 0 | 0 | 1 | 0 | 0 | 0 | 0 | 1 | 0 | 1 | 1 | 1 | 1 |
| 2 | 0 | 0 | 1 | 0 | 0 | 0 | 0 | 0 | 1 | 1 | 0 | 0 | 1 | 1 |
| 3 | 0 | 0 | 1 | 1 | 0 | 0 | 0 | 0 | 1 | 1 | 0 | 1 | 1 | 1 |
| 4 | 0 | 1 | 0 | 0 | 0 | 0 | 0 | 0 | 1 | 1 | 1 | 0 | 1 | 1 |
| 5 | 0 | 1 | 0 | 1 | 0 | 0 | 0 | 1 | 0 | 0 | 0 | 0 | 0 | 0 |
| 6 | 0 | 1 | 1 | 0 | 0 | 0 | 0 | 1 | 0 | 0 | 0 | 1 | 0 | 1 |
| 7 | 0 | 1 | 1 | 1 | 0 | 0 | 0 | 1 | 0 | 0 | 1 | 0 | 1 | 0 |
| 8 | 1 | 0 | 0 | 0 | 0 | 0 | 0 | 1 | 0 | 1 | 0 | 0 | 0 | 0 |
| 9 | 1 | 0 | 0 | 1 | 0 | 0 | 0 | 1 | 0 | 1 | 0 | 1 | 1 | 0 |
| 10 | 1 | 0 | 1 | 0 | 0 | 0 | 0 | 1 | 0 | 1 | 1 | 1 | 0 | 0 |
| 11 | 1 | 0 | 1 | 1 | 0 | 0 | 0 | 1 | 1 | 0 | 0 | 1 | 0 | 0 |
| 12 | 1 | 1 | 0 | 0 | 0 | 0 | 0 | 1 | 1 | 0 | 1 | 1 | 0 | 0 |
| 13 | 1 | 1 | 0 | 1 | 0 | 0 | 0 | 1 | 1 | 1 | 0 | 1 | 0 | 0 |
| 14 | 1 | 1 | 1 | 0 | 0 | 0 | 0 | 1 | 1 | 1 | 1 | 1 | 0 | 0 |
| 15 | 1 | 1 | 1 | 1 | 0 | 0 | 1 | 0 | 0 | 0 | 0 | 1 | 1 | 1 |

TABLE 2
Exemplary Look-up Table Corresponding to 100 KΩ

| Target Charge Value | | | | | | | | | | | | | | |
|---|---|---|---|---|---|---|---|---|---|---|---|---|---|---|
| A[3:0] | | | | | Pulse Width C[9:0] | | | | | | | | | |
| code | A₃ | A₂ | A₁ | A₀ | C₉ | C₈ | C₇ | C₆ | C₅ | C₄ | C₃ | C₂ | C₁ | C₀ |
| 0 | 0 | 0 | 0 | 0 | 0 | 1 | 1 | 0 | 0 | 0 | 0 | 0 | 0 | 1 |
| 1 | 0 | 0 | 0 | 1 | 0 | 1 | 1 | 0 | 1 | 0 | 0 | 0 | 1 | 1 |
| 2 | 0 | 0 | 1 | 0 | 0 | 1 | 1 | 1 | 0 | 0 | 0 | 1 | 1 | 0 |
| 3 | 0 | 0 | 1 | 1 | 0 | 1 | 1 | 1 | 1 | 0 | 1 | 0 | 1 | 1 |
| 4 | 0 | 1 | 0 | 0 | 1 | 0 | 0 | 0 | 0 | 1 | 0 | 0 | 0 | 1 |
| 5 | 0 | 1 | 0 | 1 | 1 | 0 | 0 | 0 | 1 | 1 | 1 | 0 | 0 | 1 |
| 6 | 0 | 1 | 1 | 0 | 1 | 0 | 0 | 1 | 1 | 0 | 0 | 0 | 1 | 1 |
| 7 | 0 | 1 | 1 | 1 | 1 | 0 | 1 | 0 | 0 | 0 | 1 | 1 | 1 | 1 |
| 8 | 1 | 0 | 0 | 0 | 1 | 0 | 1 | 0 | 1 | 1 | 1 | 1 | 1 | 0 |
| 9 | 1 | 0 | 0 | 1 | 1 | 0 | 1 | 1 | 1 | 0 | 1 | 1 | 1 | 1 |
| 10 | 1 | 0 | 1 | 0 | 1 | 1 | 0 | 0 | 1 | 0 | 0 | 0 | 1 | 0 |
| 11 | 1 | 0 | 1 | 1 | 1 | 1 | 0 | 1 | 0 | 1 | 1 | 0 | 1 | 0 |
| 12 | 1 | 1 | 0 | 0 | 1 | 1 | 1 | 0 | 0 | 1 | 0 | 1 | 0 | 0 |
| 13 | 1 | 1 | 0 | 1 | 1 | 1 | 1 | 1 | 0 | 1 | 0 | 0 | 0 | 1 |
| 14 | 1 | 1 | 1 | 0 | 1 | 1 | 1 | 1 | 1 | 1 | 1 | 1 | 1 | 1 |
| 15 | 1 | 1 | 1 | 1 | 1 | 1 | 1 | 1 | 1 | 1 | 1 | 1 | 1 | 1 |

The controller 112 is coupled to the database 111 and the measurement unit 120. According to the tissue characteristic measured by the measurement unit 120, the controller 112 selects a target look-up table from the plurality of look-up tables of the database 111 (Step S632), and converts a target charge value A to a pulse width C according to the target look-up table (Step S633). Taking Tables 1 and 2 as an example, the target charge value A has 4 bits (recorded as A[3:0]), and the pulse width C has 10 bits (recorded as C[9:0]). The respective bits of the target charge value A[3:0] are A₃, A₂, A₁, and A₀, and the respective bits of the pulse width C[9:0] are C₉, C₈, C₇, C₆, C₅, C₄, C₃, C₂, C₁, and C₀. For example, assuming the tissue characteristic measured by the measurement unit 120 represents that the impedance of the target tissue 10 plus the impedance of the electrode unit 130 is approximately 10 KΩ, then the controller 112 selects the look-up table of Table 1, for example, as the target look-up table from the plurality of look-up tables in the database 111. After selecting the target look-up table, the controller 112 can convert the target charge value A[3:0] into the pulse width C[9:0] according to the target look-up table. For example, if the target charge value A[3:0] is 9 (i.e. binary 1001), then the controller 112 can correspondingly output 86 (i.e. binary 0001010110) as the pulse width C[9:0].

A control terminal of the DPWM unit 113 is coupled to the controller 112 to receive the pulse width C. According to the pulse width C, the DPWM unit 113 generates a corresponding pulse width modulation (PWM) signal PW. The pulse width of the PWM signal PW corresponds to the pulse width C. For example, if the pulse width C has 10 bits, and the pulse width C[9:0] is 86, then the pulse width of the PWM signal PW is 86/1024 of a period. The DPWM unit 113 can be implemented by any type of digital pulse width modulator, such as a digital counter-based DPWM or a delay-line based DPWM. Since the DPWM is a known device to ones skilled in the art, further elaboration thereof is omitted hereafter.

An input terminal of the stimulation driver circuit 114 is coupled to the output terminal of the DPWM unit 113 to receive the PWM signal PW. An output terminal of the stimulation driver circuit 114 is coupled to the driver unit 130. The stimulation driver circuit 114 outputs the electrical stimulation signal Vo to the target tissue 10 through the electrode unit 130, and the stimulation driver circuit 114 controls the pulse width of the electrical stimulation signal Vo according to the PWM signal PW. The stimulation unit 110 can control the pulse width of the electrical stimulation signal Vo according to the pulse width C (Step S634).

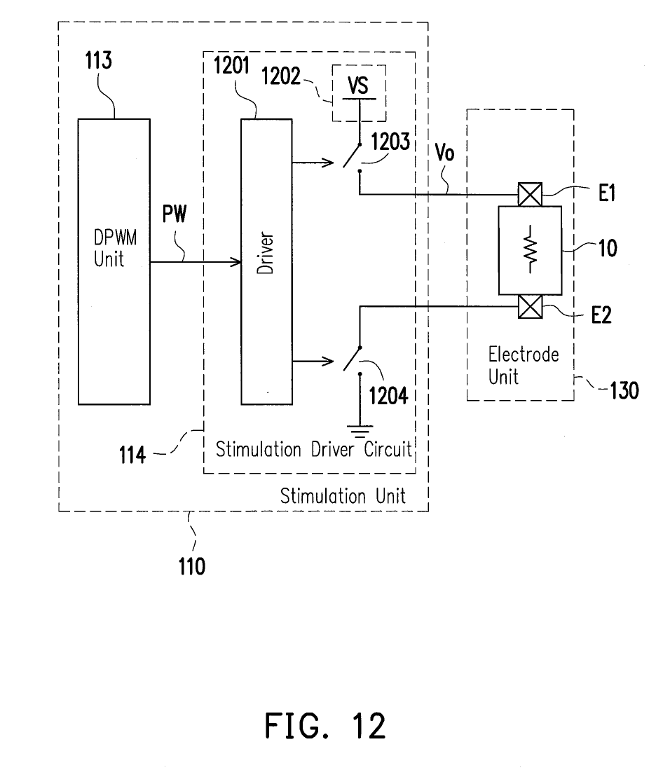

FIG. 12 is a circuit block diagram of the stimulation driver circuit 114 depicted in FIG. 10 according to an embodiment of the disclosure. The embodiment illustrated by FIG. 12 can be derived by referring to the related description of FIGS. 1-11. With reference to FIG. 12, the stimulation driver circuit 114 includes a driver 1201, a voltage source 1202, a charging switch 1203, and a discharging switch 1204. The voltage source 1202 provides a stimulation voltage VS of a fixed level. A first terminal of the charging switch 1203 is coupled to the voltage source 1202. A second terminal of the charging switch 1203 is coupled to the first electrode E1 of the electrode unit 130. A control terminal of the charging switch 1203 is coupled to a first output terminal of the driver 1201. A first terminal of the discharging switch 1204 is coupled to the ground voltage. A second terminal of the discharging switch 1204 is coupled to the second electrode E2 of the electrode unit 130. A control terminal of the discharging switch 1204 is coupled to a second output terminal of the driver 1201.

The stimulation period includes a charging period and a discharging period. A control terminal of the driver 1201 is coupled to the output terminal of the DPWM unit 113 to receive the PWM signal PW. The driver 1201 controls the charging switch 1203 according to the PWM signal PW, such that the stimulation voltage VS of the voltage source 1202 is transmitted to the target tissue 10 in the charging period through the first electrode E1 of the electrode unit 130. In the discharging period, the driver 121 controls the charging switch 1203 to turn off. The stimulation unit 110 can control the pulse width of the electrical stimulation signal Vo according to the PWM signal PW, and accumulate charge in the target tissue 10.

The driver 1201 controls the discharging switch 1204 according to the PWM signal PW, such that the ground voltage is transmitted to the target tissue 10 in the discharging period through the second electrode E2 of the electrode unit 130. The stimulation unit 110 can control the discharging switch 1204 according to the PWM signal PW, and discharge/remove the accumulated charge in the target tissue 10. In other words, during the charging period, the stimulation unit 110 couples the electrical stimulation signal Vo to the target tissue 10 through the electrode unit 130. During the discharging period, the stimulation unit 110 couples the ground voltage to the target tissue 10 through the electrode unit 130.

In view of the foregoing embodiments, an electrical stimulation apparatus and an electrical stimulation method are provided. The measuring unit 120 measures the tissue characteristic of the target tissue 10, and the tissue characteristic is fed back to the stimulation unit 110. The stimulation unit 110 can determine the pulse width of the electrical stimulation signal Vo according to the tissue characteristic measured by the measurement unit 120, and stimulate the target tissue 10 by using the electrical stimulation signal Vo with the modulated pulse width. Therefore, by configuring suitable stimulation models or look-up tables in the stimulation unit 110, the electrical stimulation apparatuses and the electrical stimulation methods according to the embodiments can satisfy any stimulus resolution (charge accuracy) requirements by using a small amount of voltage sources (or even a single level voltage source). The electrical stimulation apparatuses provided by some of the embodiments can use a voltage source to implement the voltage-controlled scenario, to meet both the high energy efficiency and low circuit area requirements.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An electrical stimulation apparatus, comprising:
an electrode unit contacting a target tissue of a biological body;
a measurement unit coupled to the electrode unit, wherein the measurement unit measures a tissue characteristic of the target tissue through the electrode unit; and
a stimulation unit coupled to the measurement unit and the electrode unit, wherein the stimulation unit stimulates the target tissue through the electrode unit by using an electrical stimulation signal, and the stimulation unit determines an amount of charge of the electrical stimulation signal according to the tissue characteristic measured by the measurement unit.

2. The electrical stimulation apparatus of claim 1, wherein the target tissue comprises a neural tissue or a cell tissue.

3. The electrical stimulation apparatus of claim 1, wherein the tissue characteristic comprises an impedance of the target tissue.

4. The electrical stimulation apparatus of claim 1, wherein the stimulation unit has a plurality of stimulation models; the stimulation models have different current-time characteristics; the stimulation unit selects a target model from the stimulation models according to the tissue characteristic measured by the measurement unit; the stimulation unit converts a target charge value into a pulse width according to the target model; and the stimulation unit controls the pulse width of the electrical stimulation signal according to the pulse width, to determine the amount of charge of the electrical stimulation signal.

5. The electrical stimulation apparatus of claim 1, wherein the stimulation unit has a plurality of look-up tables; the stimulation unit selects a target look-up table from the plurality of look-up tables according to the tissue characteristic measured by the measurement unit; the stimulation unit converts a target charge value into a pulse width according to the target look-up table; and the stimulation unit controls the pulse width of the electrical stimulation signal according to the pulse width, so as to determine the amount of charge of the electrical stimulation signal.

6. The electrical stimulation apparatus of claim 1, wherein the measurement unit measures the tissue characteristic in a measurement period through the electrode unit; and in a stimulation period after the measurement period has ended, the stimulation unit determines the pulse width of the electrical stimulation signal according to the tissue characteristic measured by the measurement unit, and stimulates the target tissue through the electrode unit by using the electrical stimulation signal.

7. The electrical stimulation apparatus of claim 6, wherein the stimulation period comprises a charging period and a discharging period; in the charging period, the stimulation unit couples the electrical stimulation signal to the target tissue through the electrode unit; and in the discharging period, the stimulation unit couples a ground voltage to the target tissue through the electrode unit.

8. The electrical stimulation apparatus of claim 1, wherein the electrode unit comprises:
a first electrode coupled to a first side of the target tissue;
a second electrode coupled to a second side of the target tissue, wherein the first side and the second side are opposite sides of the target tissue; and
a switching unit coupled to the first electrode, the second electrode, the measurement unit, and the stimulation unit;
wherein in a measurement period, the switching unit couples the first electrode and the second electrode to the measurement unit; and in a stimulation period after the measurement period has ended, the switching unit couples the first electrode and the second electrode to the stimulation unit.

9. The electrical stimulation apparatus of claim 8, wherein the switching unit comprises:
a first switch having a first terminal and a second terminal respectively coupled to the stimulation unit and the first electrode;
a second switch having a first terminal and a second terminal respectively coupled to a reference current generator of the measurement unit and the first electrode;
a third switch having a first terminal and a second terminal respectively coupled to a first input terminal of a voltage comparator of the measurement unit and the first electrode; and
a fourth switch having a first terminal and a second terminal respectively coupled to a second input terminal of the voltage comparator and the second electrode.

10. The electrical stimulation apparatus of claim 8, wherein the switching unit comprises:
a first switch having a first terminal and a second terminal respectively coupled to the stimulation unit and the first electrode;
a second switch having a first terminal and a second terminal respectively coupled to a reference current generator of the measurement unit and the first electrode;
a first conductive line having a first end and a second end respectively coupled to a first input terminal of a voltage comparator of the measurement unit and the first electrode; and
a second conductive line having a first end and a second end respectively coupled to a second input terminal of the voltage comparator and the second electrode.

11. The electrical stimulation apparatus of claim 1, wherein the electrode unit comprises:
a first electrode coupled to a first side of the target tissue;
a second electrode coupled to a second side of the target tissue, wherein the first side and the second side are opposite sides of the target tissue;
a third electrode coupled to a third side of the target tissue;
a fourth electrode coupled to a fourth side of the target tissue, wherein the third side and the fourth side are opposite sides of the target tissue; and
a switching unit coupled to the first electrode, the second electrode, the third electrode, the fourth electrode, the measurement unit, and the stimulation unit;
wherein in a measurement period, the switching unit couples the first electrode, third electrode, and the fourth electrode to the measurement unit; and in a stimulation period after the measurement period has ended, the switching unit couples the first electrode to the stimulation unit.

12. The electrical stimulation apparatus of claim 11, wherein the second electrode is coupled to a ground voltage or the stimulation unit.

13. The electrical stimulation apparatus of claim 11, wherein the switching unit comprises:
a first switch having a first terminal and a second terminal respectively coupled to the stimulation unit and the first electrode;
a second switch having a first terminal and a second terminal respectively coupled to a reference current generator of the measurement unit and the first electrode;
a third switch having a first terminal and a second terminal respectively coupled to a first input terminal of a voltage comparator of the measurement unit and the third electrode; and
a fourth switch having a first terminal and a second terminal respectively coupled to a second input terminal of the voltage comparator and the fourth electrode.

14. The electrical stimulation apparatus of claim 11, wherein the switching unit comprises:
a first switch having a first terminal and a second terminal respectively coupled to the stimulation unit and the first electrode;
a second switch having a first terminal and a second terminal respectively coupled to a reference current generator of the measurement unit and the first electrode;
a first conductive line having a first end and a second end respectively coupled to a first input terminal of a voltage comparator of the measurement unit and the third electrode; and
a second conductive line having a first end and a second end respectively coupled to a second input terminal of the voltage comparator and the fourth electrode.

15. The electrical stimulation apparatus of claim 1, wherein the measurement unit comprises:
a reference current generator coupled to the electrode unit, wherein the reference current generator provides a reference current to the target tissue through the electrode unit; and
a voltage comparator having a first input terminal and a second input terminal coupled to the electrode unit, and an output terminal coupled to the stimulation unit, wherein the first input terminal and the second input terminal of the voltage comparator are respectively coupled to a first side and a second side of the target tissue through the electrode unit.

16. The electrical stimulation apparatus of claim 1, wherein the measurement unit comprises:
a reference current generator coupled to the electrode unit, wherein the reference current generator provides a reference current to the target tissue through the electrode unit;
a voltage comparator coupled to the electrode unit, wherein a first input terminal and a second input terminal of the voltage comparator are respectively coupled to a first side and a second side of the target tissue through the electrode unit; and
an analog-to-digital converter (ADC) having an input terminal coupled to the output terminal of the voltage comparator, and an output terminal coupled to the stimulation unit.

17. The electrical stimulation apparatus of claim 16, wherein the voltage comparator comprises an instrumentation amplifier, an operation amplifier, or an error amplifier.

18. The electrical stimulation apparatus of claim 1, wherein the stimulation unit comprises:
a database having a plurality of look-up tables;
a controller coupled to the database and the measurement unit, wherein the controller selects a target look-up table from the plurality of look-up tables according to the tissue characteristic measured by the measurement unit, and converts a target charge value into a pulse width according to the target look-up table;
a digital pulse width modulation (DPWM) unit having a control terminal coupled to the controller to receive the pulse width, the DPWM unit generating a pulse width modulation (PWM) signal according to the pulse width, wherein a pulse width of the PWM signal corresponds to the pulse width; and
a stimulation driver circuit having an input terminal coupled to an output terminal of the DPWM unit to receive the PWM signal, and an output terminal coupled to the electrode unit, wherein the stimulation driver circuit outputs the electrical stimulation signal to the target tissue through the electrode unit, and the stimulation driver circuit controls the pulse width of the electrical stimulation signal according to the PWM signal.

19. The electrical stimulation apparatus of claim 18, wherein the stimulation driver circuit comprises:
a voltage source providing the electrical stimulation signal;
a charging switch having a first terminal and a second terminal respectively coupled to the voltage source and the electrode unit; and
a driver having a control terminal coupled to the output terminal of the DPWM unit to receive the PWM signal, and a first output terminal coupled to a control terminal of the charging switch;
wherein according to the PWM signal, the driver controls the charging switch so the electrical stimulation signal is transmitted in a charging period to the target tissue through the electrode unit.

20. The electrical stimulation apparatus of claim 19, wherein the stimulation driver circuit further comprises:
a discharging switch having a first terminal and a second terminal respectively coupled to a ground voltage and the electrode unit, and a control terminal coupled to a second output terminal of the driver;
wherein according to the PWM signal, the driver controls the discharging switch so the ground voltage is transmitted in a discharging period to the target tissue through the electrode unit.

21. An electrical stimulation method, comprising:
disposing an electrode unit for contacting a target tissue of a biological body;
by using a measurement unit, measuring a tissue characteristic of the target tissue through the electrode unit; and
by using a stimulation unit, stimulating the target tissue through the electrode unit by using an electrical stimulation signal, wherein an amount of charge of the electrical stimulation signal is determined by the stimulation unit according to the tissue characteristic measured by the measurement unit.

22. The electrical stimulation method of claim 21, wherein the target tissue comprises a neural tissue or a cell tissue.

23. The electrical stimulation method of claim 21, wherein the tissue characteristic comprises an impedance of the target tissue.

24. The electrical stimulation method of claim 21, wherein the step of determining the amount of charge of the electrical stimulation signal comprises:
providing a plurality of stimulation models, wherein the stimulation models have different current-time characteristics;
according to the tissue characteristic measured by the measurement unit, selecting a target model from the stimulation models;
according to the target model, converting a target charge value into a pulse width; and
controlling the pulse width of the electrical stimulation signal according to the pulse width by the stimulation unit, so as to determine the amount of charge of the electrical stimulation signal.

25. The electrical stimulation method of claim 21, wherein the step of determining the amount of charge of the electrical stimulation signal comprises:
providing a plurality of look-up tables;
according to the tissue characteristic measured by the measurement unit, selecting a target look-up table from the plurality of look-up tables;
according to the target look-up table, converting a target charge value into a pulse width; and
controlling the pulse width of the electrical stimulation signal according to the pulse width by the stimulation unit, so as to determine the amount of charge of the electrical stimulation signal.

26. The electrical stimulation method of claim 21, wherein the measurement unit measures the tissue characteristic in a measurement period through the electrode unit; and in a stimulation period after the measurement period has ended, the stimulation unit determines the pulse width of the electrical stimulation signal according to the tissue characteristic measured by the measurement unit, and stimulates the target tissue through the electrode unit by using the electrical stimulation signal.

27. The electrical stimulation method of claim 26, wherein the stimulation period comprises a charging period and a discharging period, and the step of stimulating the target tissue by using the electrical stimulation signal comprises:
by using the stimulation unit in the charging period, transmitting the electrical stimulation signal to the target tissue through the electrode unit; and
by using the stimulation unit in the discharging period, transmitting a ground voltage to the target tissue through the electrode unit.

28. The electrical stimulation method of claim 21, wherein the step of disposing the electrode unit comprises:
disposing a first electrode to a first side of the target tissue;
disposing a second electrode to a second side of the target tissue, wherein the first side and the second side are opposite sides of the target tissue;
wherein in a measurement period, the first electrode and the second electrode are coupled to the measurement unit; and in a stimulation period after the measurement period has ended, the first electrode and the second electrode are coupled to the stimulation unit.

29. The electrical stimulation method of claim 21, wherein the step of disposing the electrode unit comprises:
disposing a first electrode to a first side of the target tissue;
disposing a second electrode to a second side of the target tissue, wherein the first side and the second side are opposite sides of the target tissue;
disposing a third electrode to a third side of the target tissue; and
disposing a fourth electrode coupled to a fourth side of the target tissue, wherein the third side and the fourth side are opposite sides of the target tissue, and the third electrode and the fourth electrode are coupled to the measurement unit;
wherein in a measurement period, the first electrode is coupled to the measurement unit; and in a stimulation period after the measurement period has ended, the first electrode is coupled to the stimulation unit.

30. The electrical stimulation method of claim 29, wherein the second electrode is coupled to a ground voltage or the stimulation unit.

31. The electrical stimulation method of claim 21, wherein the step of measuring the tissue characteristic of the tissue comprises:
providing a reference current to the target tissue through the electrode unit;
comparing a voltage of a first side of the target tissue with a voltage of a second side of the target tissue, so as to obtain a comparison result; and
using the comparison result as the tissue characteristic measured by the measurement unit, and providing the tissue characteristic to the stimulation unit.
